(19) United States
(12) Patent Application Publication
(10) Pub. No.: US 2022/0168754 A1
(43) Pub. Date: Jun. 2, 2022

(54) ENHANCED CENTRIFUGE FOR CORE SAMPLE ANALYSIS

(71) Applicant: LANDMARK GRAPHICS CORPORATION, Houston, TX (US)

(72) Inventors: Travis St. George RAMSAY, Hockley, TX (US); Chuck BALDWIN, Houston, TX (US)

(73) Assignee: LANDMARK GRAPHICS CORPORATION, Houston, TX (US)

(21) Appl. No.: 17/601,875

(22) PCT Filed: May 10, 2019
(86) PCT No.: PCT/US2019/031737

§ 371 Date: Oct. 6, 2021

(51) Int. Cl. B04B 5/02; G01N 15/08

(52) U.S. Cl. CPC B04B 5/02; G01N 15/0826

(57) ABSTRACT

Disclosed herein are example configurations of centrifuges used for analyzing properties of core samples extracted from sub-surface environments, in which the configuration of the centrifuges and rotations thereof improve fluid distribution within core samples held in the apparatus. In one aspect, a centrifuge includes a rotating arm and a holder coupled to a distal end of the rotating arm, the holder being configured to rotate independently of the rotating arm for analyzing fluid-rock interaction within the holder.

### TECHNICAL FIELD

The present technology pertains to improvements in centrifuges and more particularly to centrifuges that improve distribution of injected liquids into rock samples held in such centrifuges to obtain more accurate analysis of fluid-rock interactions.

### BACKGROUND

A centrifuge is a machine/device that has a rotating arm to the end of which a sample holder and a vial are attached. The sample holder holds samples of rock, soil, etc. (core samples) obtained from subsurface extractions. Liquid is injected into the sample holder and as the rotating arm rotates, remainder of the fluid, after interaction with the core samples is collected in the vial. The collected remaining fluid can be used to study the core samples' relative permeability and capillary pressure.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrate a wellbore operating environment in which a core sampling apparatus may be deployed for extracting core samples, according to one aspect of the present disclosure;

FIG. 2 is an example depiction of a reservoir, according to one aspect of the present disclosure;

FIG. 3 illustrates an example centrifuge for analyzing core samples extracted by the core sampling apparatus of FIG. 1, according to one aspect of the present disclosure;

FIG. 4 illustrates an example centrifuge for analyzing core samples extracted by the core sampling apparatus of FIG. 1, according to one aspect of the present disclosure;

FIG. 5 illustrates an example centrifuge for analyzing core samples extracted by the core sampling apparatus of FIG. 1, according to one aspect of the present disclosure;

FIG. 6 illustrates an example centrifuge for analyzing core samples extracted by the core sampling apparatus of FIG. 1, according to one aspect of the present disclosure; and

FIG. 7 is an example method of analyzing extracted core samples, according to one aspect of the present disclosure.

### DETAILED DESCRIPTION

Various example embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

It will be appreciated that for simplicity and clarity of illustration, where appropriate, reference numerals have been repeated among the different figures to indicate corresponding or analogous elements. In addition, numerous specific details are set forth in order to provide a thorough understanding of the example embodiments described herein. However, it will be understood by those of ordinary skill in the art that the example embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the example embodiments described herein.

Conventional centrifuges may involve the uneven distribution of fluid, thereby resulting in uneven saturation in the core samples. Because the centrifugal force on the core samples differs at each segment of the core samples (as the rotating arm rotates) depending on each segment's relative distance to the axis of rotation of the rotating arm. Furthermore, fluid distributions in the core sample are axially variable about the longitudinal axis of the sample holder due to the core sample being stationary in the plane orthogonal to the axis of rotation of the rotating arm. Due to such uneven distributions, when corresponding relative permeability curves are utilized in subsequent simulations of reservoirs to conduct a history match or forecast production, the ensuing predictions and business decisions for resource extractions will be less accurate and suboptimal.

Disclosed herein are example configurations of centrifuges used for analyzing properties of core samples extracted from sub-surface environments, in which the configuration of the centrifuges and rotations thereof improve fluid distribution within core samples held in the apparatus. The disclosure begins with an example structure for extracting core samples.

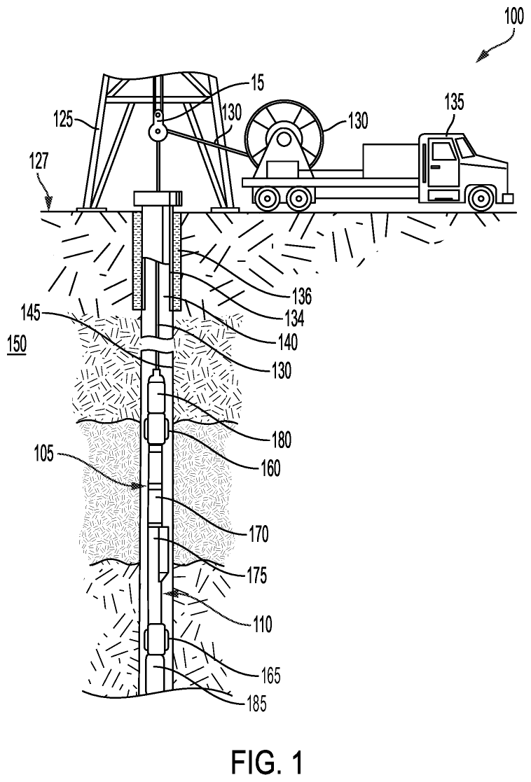

FIG. 1 illustrate a wellbore operating environment in which a core sampling apparatus may be deployed for extracting core samples, according to one aspect of the present disclosure. As depicted, operating environment 100 includes a derrick 125 that supports a hoist 115. Drilling oil and gas wells is commonly carried out using a string of drill pipes connected together so as to form a drilling string that is lowered through a rotary table into a wellbore 140. Here it is assumed that the drill string has been temporarily removed from wellbore 140 to allow a downhole core sampling apparatus 105 to be lowered into wellbore 140 that has been previously drilled through one or more formations 150.

As depicted, downhole core sampling apparatus 105 can be lowered into wellbore 140 by conveyance 130 coupled with hoist 115, A casing 134 can be secured within wellbore 140 by cement 136. Conveyance 130 can be anchored to derrick 125 or portable or mobile units such as a true 135. Conveyance 130 provides support for downhole core sampling apparatus 105, as well as enabling communication between downhole core sampling apparatus 105 and processors or controllers at surface 127 outside wellbore 140. Conveyance 130 can be one or more wires, wireline, slickline, cables, tubulars, coiled tubing, joint tubing, or the like. Conveyance 130 can include fiber optic cabling or other wire or cable for carrying, out communications. An optical cable can be provided internal or external of conveyance 130. Conveyance 130 is sufficiently strong and flexible to tether downhole core sampling apparatus 105 through wellbore 140, while also permitting communication through the conveyance 130 to processors or controllers at surface 127, Additionally, power can be supplied via conveyance 130 to meet the power requirements of downhole core sampling apparatus 105. Downhole core sampling apparatus 105 can have a local power supply, such as batteries, downhole generator and the like. When employing non-conductive cable, coiled tubing, pipe string, or downhole tractor, communication may be supported using, for example, wireless protocols (e.g. EM, acoustic, etc.), and/or measurements and logging data may be stored in local memory for subsequent retrieval. While a conveyance 130 is illustrated in FIG. 1, other conveyances may, be used to convey core sampling apparatus 105 into the wellbore. In some instances, core sampling apparatus 105 can be conveyed by wired coiled tubing.

As depicted in FIG. 1, downhole core sampling apparatus 105 is lowered into wellbore 140 penetrating one or more formations 150 to a desired core sampling zone after which downhole core sampling apparatus 105 may sample cores from sidewall 145 of wellbore 140. Core sampling apparatus 105 can include an elongate housing 110, a first sealing element 160, a second sealing element 165, a sidewall coring tool 170, a core storage assembly 175, first pump 180, and second pump 185.

While FIG. 1 depicts a first sealing element 160 and a second sealing element 165, a downhole core sampling apparatus 105 that includes only a single sealing element is within the spirit and scope of the present disclosure. For instance, downhole core sampling apparatus 105 may include only first sealing element 160. In other examples, downhole core sampling apparatus 105 may include only second sealing element 165.

Although FIG. 1 depicts a vertical wellbore 140, the present disclosure is equally well-suited for use in wellbores having other orientations including horizontal wellbores, slanted wellbores, multilateral wellbores or the like. Also, even though FIG. 1 depicts an onshore operation, the present disclosure is equally well-suited for use in offshore operations.

FIG. 1 illustrates just one example embodiment of a wellbore operating environment in which a downhole core sampling apparatus, method, and system may be deployed. The core sampling apparatus, method, and system may be deployed in other operating environments, such as a drilling environment. For instance, core sampling apparatus 105 may be placed in a wellbore as part of a Measurement while drilling (MWD) portion of a drillstring or as part of a logging while drilling (LWD) portion of a drillstring. In other instances, the core sampling apparatus 105 may be on a drillpipe as part of a wired drillpipe system.

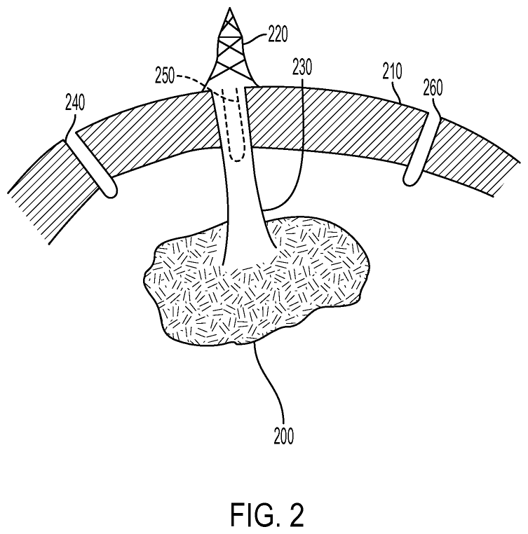

Core samples extracted/collected by core sample apparatus 105 may be transferred to an onsite or offsite laboratory setting in which the collected core samples may be tested/analyzed to better understand various subsurface properties and formation 150. The results of such testing/analysis of core samples can be used for generating earth models, reservoir simulations and perforation simulation, which provide an insight on formation and resource production potential of the site(s) from which the core samples have been extracted. Examples of resources include but are not limited to various hydrocarbons (e.g., oil, natural gas, etc.), minerals, natural substances (e.g., gold, copper, etc.), water, etc. An example of a reservoir with a sub-surface oilfield is shown in FIG. 2.

FIG. 2 is an example depiction of a reservoir, according to one aspect of the present disclosure. As shown in FIG. 2, a hydrocarbon reservoir 200 exists beneath surface 210. FIG. 2 also illustrates oil rig 220 installed on surface 210 for extracting the hydrocarbon from reservoir 200. Prior to drilling well 230 for reaching reservoir 200 and because the actual location of reservoir 200 and its potential production of hydrocarbon are unknown, several wellbores 240, 250 and 260 may be drilled, each of which may be the same as wellbore 140 and sampled using downhole core sampling apparatus of FIG. 1.

As noted, extracted core samples may then be taken to a laboratory for further analysis and studies of properties of extracted core samples, which are then used to determine whether actual wells for hydrocarbon extraction should be constructed at any of the wellbores 240, 250 and 260. In one example, after such studies and completion of the ensuing reservoir simulation, a business decision may be made to build well 230 where wellbore 250 was originally drilled. Additionally, a perforation can be planned so that a physical completion can be developed.

Various apparatuses and methods exist for testing an analyzing the core samples. As mentioned above, one example apparatus is a centrifuge with a rotating arm to an end of which a sample holder and a vial is connected to determine fluid-rock interaction. As also noted, the uneven distribution of fluid in current settings of such centrifuges, as the centrifuge rotates, provides a suboptimal accuracy in determining relative permeability and capillary pressure curves. Hereinafter, example embodiments providing an improved centrifuge apparatus to address the problem of uneven distribution of fluid as the centrifuge rotates, will be described.

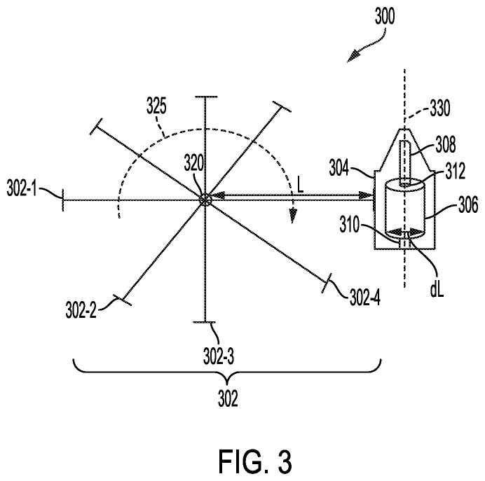

FIG. 3 illustrates an example centrifuge for analyzing core samples extracted by the core sampling apparatus of FIG. 1, according to one aspect of the present disclosure. As shown in FIG. 3, apparatus (centrifuge) 300 includes a rotary portion 302. Rotating portion 302 may have several rotating arms such as arms 302-1, 302-2, 302-3 and 302-4. The number of rotating arms of rotating portion 302 is not limited to 4 but may be more or less and can be a matter of design choice and practicality of centrifuge 300. Arms 302-1, 302-2, 302-3 and 302-4 may be made of any type of known or to be developed material suitable for core sample testing such as plastic, metal, glass, etc.

To a distal end of rotating arm 302-1, a holder 304 may be attached. Holder 304 may be made of any type of known or to be developed material suitable for core sample testing such as plastic, metal, glass, etc. Holder 304 may include a physical core 306 and a vial 308.

Physical core 306 may have a cylindrical shape or any other shape that allows holder 306 to rotate within holder 304. Physical core 306 may be made of any type of known or to be developed material suitable for core sample testing such as plastic, metal, glass, etc. Physical core 306 may also be referred to as sample physical core 306 and may hold samples of rocks, soils, etc. extracted from wellbore 140, 240, 250 and/or 260 of FIGS. 1 and 2. Orientation of Physical core 306 may be perpendicular to axis of rotation of rotating portion 302 and tangential to angular direction 325 which is the rotation direction of rotating arm 302.

Physical core 306 may have openings 310 and 312. Liquid may be injected into physical core 306 via opening 310 and after interaction with core samples in physical core 306, at least a portion of the liquid may exit physical core 306 through opening 312 and into vial 308. One or more of openings 310 and 312 may be sealable (openly closable).

Vial 308 may be made of any type of known or to be developed material suitable for core sample testing such as plastic, metal, glass, etc. Vial 308 may collect any remaining amount of liquid, after interaction with core sample inside physical core 306, based on which relative permeability and/or capillary pressure of core samples inside physical core 306 may be determined and analyzed, using known or to be developed methods for doing so.

FIG. 3 illustrates further illustrates center of rotation 320 of rotary portion 302, which may be the same as center of rotation of rotation of rotating arm 302-1. Axis of rotation of rotary portion 302 runs through center of rotation 320 (coming out of/going into the paper when looking at FIG. 3 from above and perpendicular to orientation of holder 304. Rotating arm 302-1 and more generally rotating portion 302 rotates alone angular direction 325. Rotating portion's rotation is not limited to angular direction 325 and can, for example, be in the opposite of angular direction 325.

In configuration of centrifuge 300 and in order to address the problem of uneven distribution of liquid inside holder 304 as rotating arm 302-1 rotates, physical core 306 (or alternatively holder 304) may rotate independently of (and/or simultaneously with) rotation of rotating arm 302-1, where an axis of rotation of physical core 306 is axis 330 that is perpendicular to axis of rotation of rotating portion 302.

Additionally, in configuration of centrifuge 300, a distance from center of rotation 320 to a distal end of rotating arm 302-1 to which holder 304 is attached may have length L. Furthermore, width of physical core 306 may have length dL. In one example, length L may be sufficiently larger than dL (L>>dL) so that a distance from center of rotation 320 to any point within physical core 306 may be approximately the same (i.e., less than a threshold, where the value of the threshold may be a design parameter determined based on experiments and/or empirical studies). For example, length L may be sufficiently larger than dL so that a first distance from center of rotation 320 to any point within physical core 306 may deviate from a second distance from center of rotation 320 to any other point within physical core 306, by no more than 2%, 5%, etc.

Using configuration of centrifuge 300, capillary pressure of core samples inside physical core 306 can be determined partly based on equation (1) instead of equation (2) used in conjunction with currently available centrifuge settings (where orientation of holder 304 is along the length of rotating arm 302-1 (perpendicular to angular rotation 325) and the only rotating portion is the arm 302-1):

$$\begin{matrix}{P}_{c} = \frac{1}{2} Δ ρ {ω}^{2} ( {L}^{2} ) & ( 1 ) \\ {P}_{c} = \frac{1}{2} Δ ρ {ω}^{2} ( {L}^{2} - d {L}_{i}^{2} ) & ( 2 )\end{matrix}$$

With L being sufficiently larger than dL, dL; can be effectively ignored from equation (2) to arrive at equation (1). Each of equations (1) and (2) may have additional terms for each distance from center of rotation 320 to a given point in physical core 306. Equation (1) may then be used in any known or to be developed method of determining capillary pressure or relative permeability to determine the same. As example of such method is given in Kantzas et al., “Two Phase Relative Permeabilities Using Ultracentrifuge,” 1995, 58-63, the entire content of which is incorporated herein by reference.

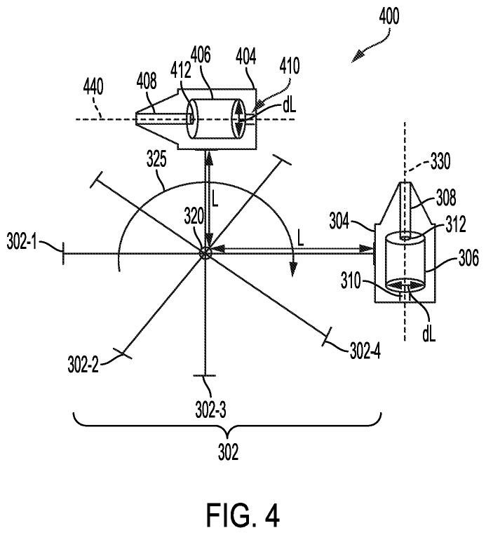

In FIG. 3, while rotating portion 302 is shown as having multiple rotating arms 302-1, 302-2, 302-3 and 302-4, only one rotating arm (arm 302-1) is shown having holder 304 attached to a distal end thereof. However, it is possible to have multiple holders attached to multiple rotating arms of rotating portion 302. An example of such setting is shown in FIG. 4.

FIG. 4 illustrates an example centrifuge for analyzing core samples extracted by the core sampling apparatus of FIG. 1, according to one aspect of the present disclosure. Centrifuge 400 is an example, which includes all components of centrifuge 300 and thus such components will not be repeated for sake of brevity. Furthermore and in contrast to centrifuge 300, centrifuge 400 can also have one additional holder 404 attached to another rotating arm 302-3 such that two simultaneous analysis of fluid-rock interaction can be carried out during a single use of centrifuge 400. Holder 404 is the same as holder 304 and will not be described further for sake of brevity. Physical core 406 is the same as physical core 306 and will not be described further for sake of brevity. Vial 408 is the same as vial 308 and will not be described further for sake of brevity. Opening 410 is the same as opening 310 and will not be described further for sake of brevity. Opening 412 is the same as opening 312 and will not be described further for sake of brevity. Axis of rotation 430 of physical core 406 is perpendicular to axis of rotation of rotating portion 302 and hence rotating arm 302-2 and will not be described further for sake of brevity.

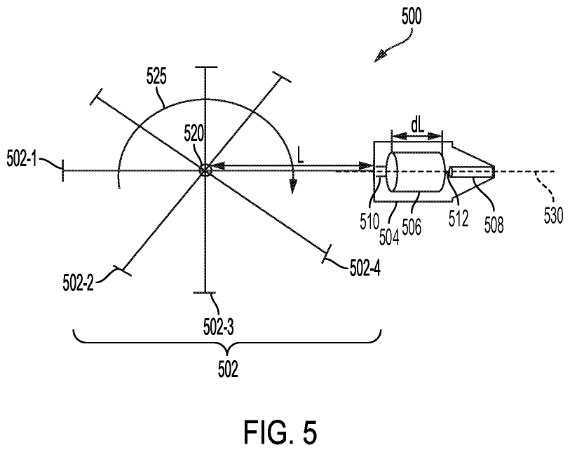

FIG. 5 illustrates an example centrifuge for analyzing core samples extracted by the core sampling apparatus of FIG. 1, according to one aspect of the present disclosure. FIG. 5 illustrates another example centrifuge 500.

Rotating portion 502 is the same as rotating portion 302 of FIG. 3 and will not be described further. Rotating arms 502-1, 502-2, 502-3 and 502-4 are the same rotating arms 302-1, 302-2, 302-3 and 302-4, respectively, and will not be described further for sake of brevity. Center of rotation 520 and angular direction 525 are the same as center of rotation 320 and angular direction 325, respectively and will not be described further for sake of brevity.

Furthermore, in FIG. 5, holder 504 is the same as holder 304 and will not be described further for sake of brevity. Physical core 506 is the same as physical core 506 and will not be described further for sake of brevity. Vial 408 is the same as vial 308 and will not be described further for sake of brevity. Opening 510 is the same as opening 310 and will not be described further for sake of brevity. Opening 512 is the same as opening 312 and will not be described further for sake of brevity.

In contrast to centrifuge 300 of FIG. 3, for centrifuge 500, a distance L from center of rotation 520 of rotating portion 502 to a distal end of rotating arm 502-1 to which holder 504 is attached is less than the distance L of FIG. 3. Furthermore, distance dL in FIG. 5 is defined as a length of physical core 506 as opposed to width of physical core 306 in FIG. 3. Accordingly, ratio of L to dL is larger for centrifuge 500 compared to the ratio of L to dL of centrifuge 300.

Another difference between centrifuge 500 of FIG. 5 and centrifuge 300 of FIG. 3 is that axis of rotation of physical core 506 (along which physical core 506 rotates independently and/or simultaneous with rotation of rotating arm 502-1) is along a length of rotating arm 504-1 (in other words axis of rotation 530 is perpendicular to angular direction 525 as opposed to being tangential thereto as is the case with axis of rotation 330 and angular direction 325 of FIG. 3).

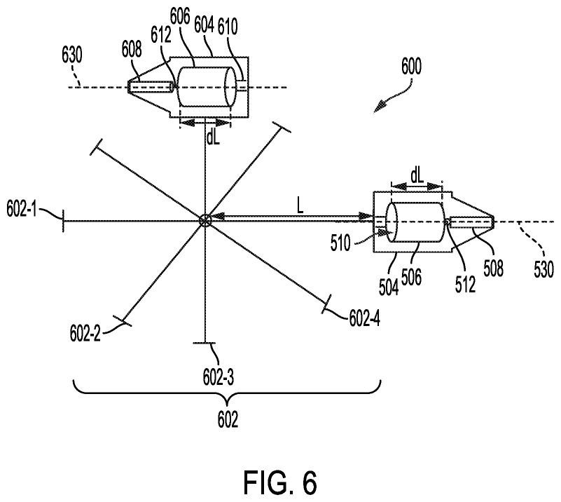

Similar to FIG. 3, in FIG. 3, rotating portion 502 is shown as having multiple rotating arms 502-1, 502-2, 502-3 and 502-4, while only one rotating arm (arm 502-1) is shown having holder 504 attached to a distal end thereof. However, it is possible to have multiple holders attached to multiple rotating arms of rotating portion 502. An example of such setting is shown in FIG. 6.

FIG. 6 illustrates an example centrifuge for analyzing core samples extracted by the core sampling apparatus of FIG. 1, according to one aspect of the present disclosure. Centrifuge 600 is an example, which includes all components of centrifuge 500 and thus such components will not be described further for sake of brevity. Furthermore and in contrast to centrifuge 500, centrifuge 600 can also have one additional holder 604 attached to another rotating arm 502-3 such that two simultaneous analysis of fluid-rock interaction can be carried out during a single use of centrifuge 600. Holder 604 is the same as holder 504 and will not be described further for sake of brevity. Physical core 606 is the same as physical core 506 and will not be described further for sake of brevity. Vial 608 is the same as vial 508 and will not be described further for sake of brevity. Opening 610 is the same as opening 510 and will not be described further for sake of brevity. Opening 612 is the same as opening 512 and will not be described further for sake of brevity. Axis of rotation 630 of physical core 606 is perpendicular to axis of rotation of rotating portion 502 and hence rotating arm 502-2. Therefore, axis of rotation 630 will not be described further for sake of brevity.

In examples configurations of FIGS. 3-6, a vial is described in which remainder of fluid after interaction with core samples inside physical cores is collected and measured for determination of saturation for relative permeability and capillary pressure analysis of the core sample. In another example embodiment, instead of a vial, magnetic resonance imaging (MRI) can be used to measure fluid saturation in the core sample.

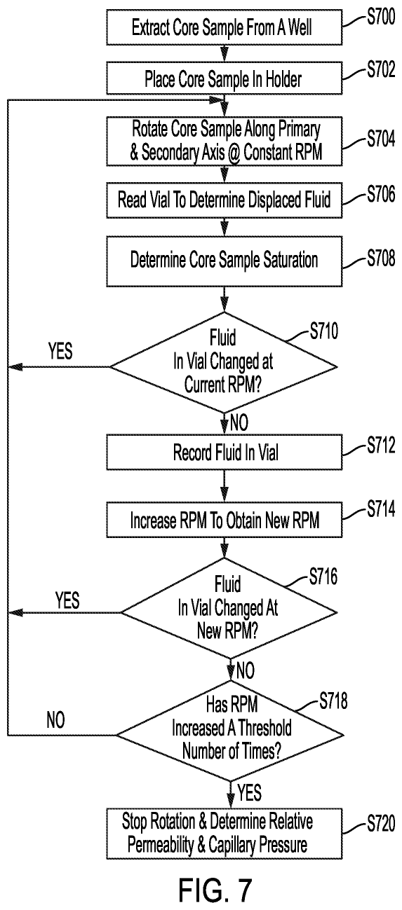

FIG. 7 is an example method of analyzing extracted core samples, according to one aspect of the present disclosure. FIG. 7 describes a method for analyzing core samples using one or more centrifuges 300, 400, 500, or 600 of FIGS. 3-6. However, for purposes of discussion, FIG. 7 will be described from perspective of centrifuge 300.

At S700, core samples may be extracted from wellbore 140 via downhole core sampling apparatus 105. As described above, such core samples may be extracted from side walls as well as depth of wellbore 140 and properties thereof analyzed for purposes of creating an earth model, reservoir simulation and perforation design for ultimate determination of viability of creating a well for hydrocarbon extraction at a location of wellbore 140.

At S702, extracted core samples may be placed inside physical core 306 of centrifuge 300.

At S704, centrifuge 300 starts to rotate at a constant speed (revolution per minute (RPM)). Rotation of centrifuge 300 may include separate rotation of rotating arm 302-1 along axis of rotation of rotating portion 302 (primary axis of rotation) as well as rotation of physical core 306 along axis of rotation 330 of physical core 306 (secondary axis of rotation). In one example, constant RPM along primary and secondary axes of rotation may be the same. In another example, constant RPM along primary axis of rotation may be different from constant RPM along secondary axis of rotation.

At S706, amount of fluid in vial 308 is read to determine the amount of fluid displaced within physical core 306. In addition to determining the amount of displaced fluid, at S706, rate of influx and/or out flux of core samples for relative permeability and pressure in the displacing phase as well as displaced phase for capillary pressure may be determined.

At S708, core sample saturation is determined based on the reading at S704, according to any known or to be developed method of reading and determining core sample saturations.

At S710, a determination is made as to whether the collected fluid in vial 308, as read at S706 changes at the current RPM. The change may be observed over a period of time duration of which may be a configurable parameter determined based on experiments and/or empirical studies.

If the fluid vial 308 changed at S710, the process returns to S704 and S704 to S710 are repeated until no changes in amount of fluid in vial 308 are observed at S710. Then, at S712, the current amount of fluid in vial 308 as observed at S708 is recorded.

At S714, RPM along primary axis of rotation and/or along secondary axis of rotation are increased by a threshold amount, where the threshold amount may be a configurable parameter determined based on experiments and/or empirical studies.

At S716, a determination is made as to whether fluid amount in vial 308 is changed at new RPM (which is the new RPM set at S714). The amount of changes may be compared to a threshold, which may be a configurable parameter determined based on experiments and/or empirical studies. If the amount of fluid is changed, the process reverts back to S704 and S704 to S716 are repeated until no change in the amount of fluid is determined at S716. In one example, the RPM may need to be increased multiple times before displacement occurs due to the pore throats of the rock and surface tension of the fluid. As a result, stopping the rotation due to lack of change in displaced fluid in the vial may be premature. This needs to be reflected in the diagram. If the amount of fluid is not changed, then at S718, a determination is made as to whether the RPM has been increased at least for a threshold number of times (where the threshold number of times is determined based on experiments and/or empirical studies). If the RPM has not been increased for at least the threshold number of times, the process reverts back to S704 and S704 to S718 are repeated until either a change in the amount of fluid in vial 308 is determined or the RPM is increased for the threshold number of times. Thereafter, at, at S720 centrifuge 300's rotation along primary and/or secondary axes of rotation are terminated and various rock properties including relative permeability and/or capillary pressure are determined for use in further reservoir simulations as noted above. Relative permeability can be determined by capturing the rate of two phase fluid flux into and out of physical core 306. Capillary pressure can be determined by capturing the injection pressure of the single phase displacing fluid and determining the pressure of the displaced fluid from physical core 306.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

### STATEMENTS OF THE DISCLOSURE INCLUDE

Statement 1: A centrifuge includes a rotating arm and a holder coupled to a distal end of the rotating arm, the holder being configured to rotate independently of the rotating arm for analyzing fluid-rock interaction within the holder.

Statement 2: The centrifuge of statement 1, wherein an axis of rotation of the holder is perpendicular to the rotating arm and tangential to direction of rotation of the rotating arm.

Statement 3: The centrifuge of statement 2, wherein a distance from a center of rotation of the rotating arm to the distal end of the rotating arm is larger than a width of the holder such that a first radial distance from the center of rotation of the rotating arm to any point in the holder differs from a second radial distance from the center of rotation of the rotating arm to any other point in the holder by less than a threshold.

Statement 4: The centrifuge of statement 1, wherein an axis of rotation of the holder is parallel to an axis of rotation of the rotating arm and perpendicular to direction of rotation of the rotating arm.

Statement 5: The centrifuge of statement 4, wherein a distance from a center of rotation of the rotating arm to the distal end of the rotating arm differs from a length of the holder by less than a threshold.

Statement 6: The centrifuge of statement 15, further including a vial configured to collect remainders of fluid exiting the holder as the rotating arm and the holder rotate for analyzing the fluid-rock interaction.

Statement 7: The centrifuge of statement 1, wherein analyzing the fluid-rock interaction includes measuring relative permeability and capillary pressure of rock samples in the holder.

Statement 8: The centrifuge of statement 1, wherein the holder has a cylindrical shape.

Statement 9: The centrifuge of statement 1, wherein a rotational speed of the rotating arm and a rotational speed of the holder are the same.

Statement 10: The centrifuge of statement 1, wherein a rotational speed of the rotating arm and a rotational speed of the holder are different.

Statement 11: A centrifuge of statement 1 includes a rotating arm configured to rotate around a first axis of rotation, a holder coupled to a distal end of the rotating arm, the holder being configured to rotate independently of the rotating arm around a second axis of rotation, wherein the holder is configured to hold core samples and allow fluid to interact with the core samples as the rotating arm and the holder rotate, and a vial configured to collect measurable amount of the fluid, after interaction with the core samples for analyzing fluid-rock interaction of core samples within the holder.

Statement 12: The centrifuge of statement 11, wherein the first axis of rotation is perpendicular to the second axis of rotation and tangential to direction of rotation of the rotating arm.

Statement 13: The centrifuge of statement 12, wherein a distance from the first axis of rotation of the rotating arm to the distal end of the rotating arm is larger than a width of the holder such that a first radial distance from the center of rotation of the rotating arm to any point in the holder differs from a second radial distance from the center of rotation of the rotating arm to any other point in the holder by less than a threshold.

Statement 14: The centrifuge of statement 11, wherein the first axis of rotation is perpendicular to direction of rotation of the rotating arm.

Statement 15: The centrifuge of statement 14, wherein a distance from a center of rotation of the rotating arm to the distal end of the rotating arm differs from a length of the holder by less than a threshold.

Statement 16: The centrifuge of statement 11, wherein analyzing the fluid-rock interaction includes measuring relative permeability and capillary pressure of the core samples in the holder.

Statement 17: The centrifuge of statement 11, wherein the holder has a cylindrical shape.

Statement 18: The centrifuge of statement 11, wherein a rotational speed of the rotating arm and a rotational speed of the holder are the same.

Statement 19: The centrifuge of statement 11, wherein a rotational speed of the rotating arm and a rotational speed of the holder are different.

Statement 20: The centrifuge of statement 11, wherein a result of analyzing fluid-rock interaction of the core samples are used for one or more reservoir simulation and/or wellbore perforation design.

Statement 21: A method includes extracting core samples from a wellbore for determining viability of drilling a well for hydrocarbon extraction; placing the core samples in a holder of a centrifuge, the holder being coupled to a distal end of a rotating arm and configured to rotate independently of the rotating arm; rotating the rotating arm around a first axis of rotation, simultaneously with rotating the rotating arm around the first axis, rotating the holder around a second axis of rotation that is different than the first axis of rotation; injecting fluid into the holder to interact with the core samples; and analyzing core fluid interaction of the core samples based on measuring amount of fluid collected in a vial of the centrifuge after passing through the core samples inside the holder.

1. A centrifuge, comprising:
a rotating arm; and
a holder coupled to a distal end of the rotating arm, the holder being configured to rotate independently of the rotating arm for analyzing fluid-rock interaction within the holder.

2. The centrifuge of claim 1, wherein an axis of rotation of the holder is perpendicular to the rotating arm and tangential to direction of rotation of the rotating arm.

3. The centrifuge of claim 2, wherein a distance from a center of rotation of the rotating arm to the distal end of the rotating arm is larger than a width of the holder such that a first radial distance from the center of rotation of the rotating arm to any point in the holder differs from a second radial distance from the center of rotation of the rotating arm to any other point in the holder by less than a threshold.

4. The centrifuge of claim 1, wherein an axis of rotation of the holder is parallel to an axis of rotation of the rotating arm and perpendicular to direction of rotation of the rotating arm.

5. The centrifuge of claim 4, wherein a distance from a center of rotation of the rotating arm to the distal end of the rotating arm differs from a length of the holder by less than a threshold.

6. The centrifuge of claim 1, further comprising:
a vial configured to collect remainders of fluid exiting the holder as the rotating arm and the holder rotate for analyzing the fluid-rock interaction.

7. The centrifuge of claim 1, wherein analyzing the fluid-rock interaction includes measuring relative permeability and capillary pressure of rock samples in the holder.

8. The centrifuge of claim 1, wherein the holder has a cylindrical shape.

9. The centrifuge of claim 1, wherein a rotational speed of the rotating arm and a rotational speed of the holder are the same.

10. The centrifuge of claim 1, wherein a rotational speed of the rotating arm and a rotational speed of the holder are different.

11. A centrifuge, comprising:
a rotating arm configured to rotate around a first axis of rotation;
a holder coupled to a distal end of the rotating arm, the holder being configured to rotate independently of the rotating arm around a second axis of rotation, wherein the holder is configured to hold core samples and allow fluid to interact with the core samples as the rotating arm and the holder rotate; and
a vial configured to collect measurable amount of the fluid, after interaction with the core samples for analyzing fluid-rock interaction of core samples within the holder.

12. The centrifuge of claim 11, wherein the first axis of rotation is perpendicular to the second axis of rotation and tangential to direction of rotation of the rotating arm.

13. The centrifuge of claim 12, wherein a distance from the first axis of rotation of the rotating arm to the distal end of the rotating arm is larger than a width of the holder such that a first radial distance from the center of rotation of the rotating arm to any point in the holder differs from a second radial distance from the center of rotation of the rotating arm to any other point in the holder by less than a threshold.

14. The centrifuge of claim 11, wherein the first axis of rotation is perpendicular to direction of rotation of the rotating arm.

15. The centrifuge of claim 14, wherein a distance from a center of rotation of the rotating arm to the distal end of the rotating arm differs from a length of the holder by less than a threshold.

16. The centrifuge of claim 11, wherein analyzing the fluid-rock interaction includes measuring relative permeability and capillary pressure of the core samples in the holder.

17. The centrifuge of claim 11, wherein the holder has a cylindrical shape.

18. The centrifuge of claim 11, wherein a rotational speed of the rotating arm and a rotational speed of the holder are the same.

19. The centrifuge of claim 11, wherein a rotational speed of the rotating arm and a rotational speed of the holder are different.

20. (canceled)

21. A method comprising:
extracting core samples from a wellbore for determining viability of drilling a well for hydrocarbon extraction;
placing the core samples in a holder of a centrifuge, the holder being coupled to a distal end of a rotating arm and configured to rotate independently of the rotating arm;
rotating the rotating arm around a first axis of rotation,
simultaneously with rotating the rotating arm around the first axis, rotating the holder around a second axis of rotation that is different than the first axis of rotation;
injecting fluid into the holder to interact with the core samples; and
analyzing core fluid interaction of the core samples based on measuring amount of fluid collected in a vial of the centrifuge after passing through the core samples inside the holder.
